(19) United States
(12) Patent Application Publication
(10) Pub. No.: US 2004/0087922 A1
(43) Pub. Date: May 6, 2004

(54) Method of making early indicator color changing diaper or plastic color changing training pants

(76) Inventor: Tory Leigh Bobadilla, Orlando, FL (US)

Correspondence Address: Tory Leigh Bobadilla, 11048 LEDGEMENT LANE, WINDERMERE, FL, 34786, US

(21) Appl. No.: 10/286,995

(22) Filed: Nov. 4, 2002

(51) Int. Cl. A61F 13/15

(52) U.S. Cl. 604/361

(57) ABSTRACT

The product includes a material on the outside front of the diaper/training pant that allows color to be seen thru it from outside the diaper. Behind this material will be litmus paper or a color changing ink. Behind this litmus paper or color changing ink will be the urine absorbing material. The intent being that when the urine has sufficiently passed the layer of urine absorbing material and makes contact with the litmus paper or color changing ink a reaction will occur that will change the color of the litmus paper or the color changing ink. This color change would be visible from outside the diaper indicating that the diaper has been soiled.

### FIELD OF THE INVENTION

The present invention is directed to a method of detecting when a person wearing a diaper has soiled it with urine.

### BACKGROUND OF THE INVENTION

Presently diapers are worn mostly by small children and the elderly. Many times when the person wearing the diaper contaminates it with urine, they are unable to alert the person responsible of changing the diaper that it is dirty. This is because small children lack the vocabulary and the elderly suffer from dementia, Alzheimer's, and other elderly diseases. The time-lapse between the wetting of the diaper and the discovery by the changer that is wet is a cause of diaper rash and sores for the wearer.

### A BRIEF DESCRIPTION OF THE INVENTION

This invention is generally based on a chemical method where a small piece of the diaper changes color when it experiences a change in temperature and/or becomes wet from the urine.

In one embodiment of the present invention, a portion of litmus paper is placed on the inside of the diaper at a place where it is likely to become wet when the wearer urinates. FIG. 2

Another portion of the litmus paper will be positioned and enclosed such that it is visible inside the diaper, to the person responsible for changing the diaper. FIG. 1

In another embodiment of the present invention, the litmus paper will change colors when it becomes wet with urine notifying the changer that the diaper is wet. FIG. 1

In another embodiment of the present invention, thermochromic printing ink or any color changing ink is applied to a portion of the diaper. FIG. 2

The color of the thermochromic printing ink will change colors or leave a message when the urine changes the temperature of the ink. FIG. 1

### BRIEF DESCRIPTION OF THE DRAWINGS

FIG. 1 is the front of the diaper where the color changing design will be identified.

FIG. 2 is the inside of the diaper where the litmus paper will be placed.

FIG. 3 is the front of the plastic training pants where the color changing will be identified through the clear plastic pouch.

FIG. 4 is showing the inside view of the plastic training pants. It is showing the small granules in the clear plastic pouch, where the urine will interact with the litmus paper to cause a color change.

FIG. 5 is showing the outside view of the clear plastic pouch. The litmus paper will be inserted in between the two clear pieces of the plastic pouch.

### DETAILED DESCRIPTION OF THE INVENTION

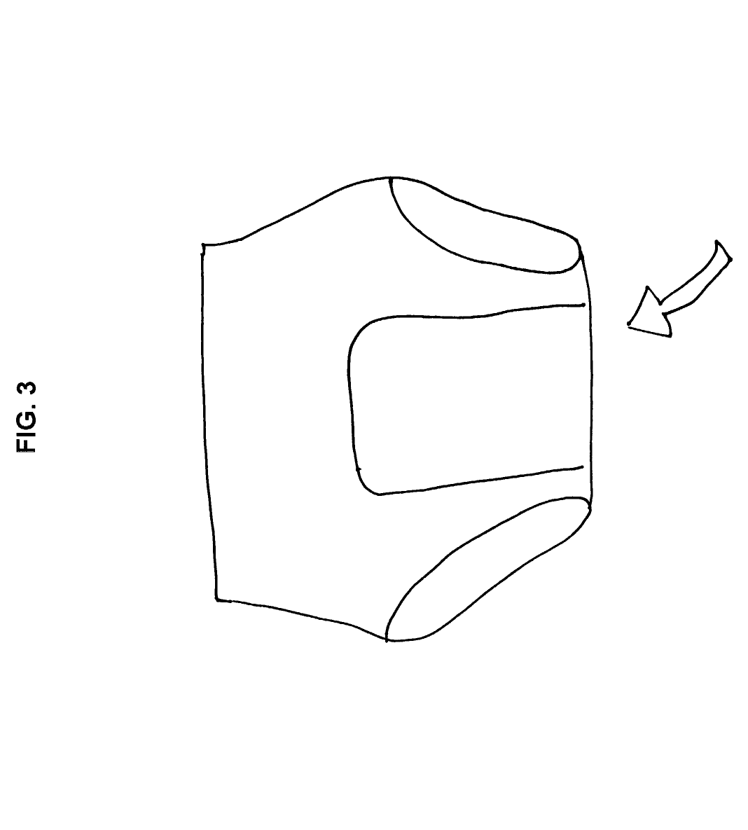

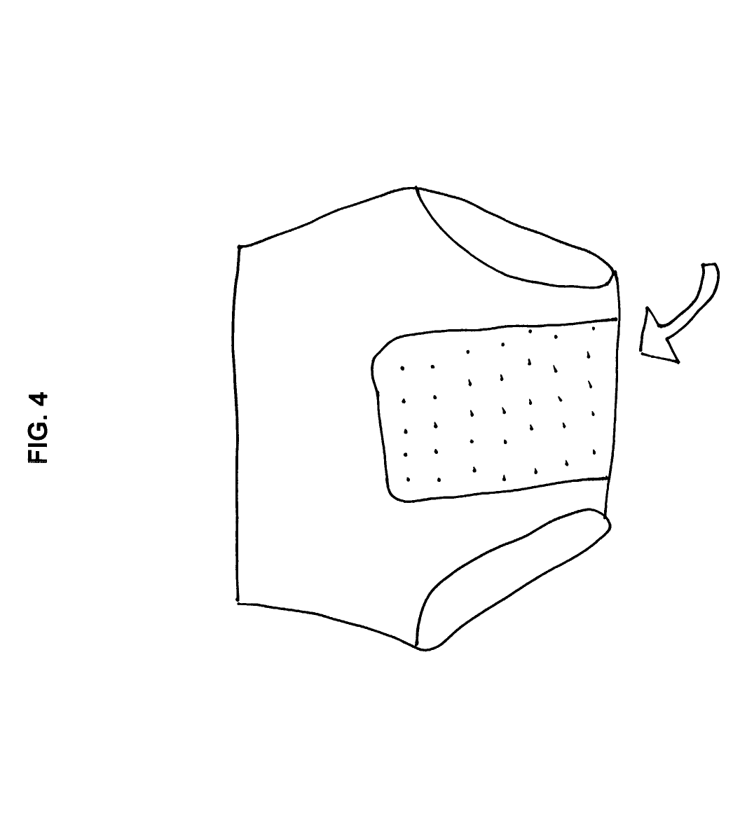

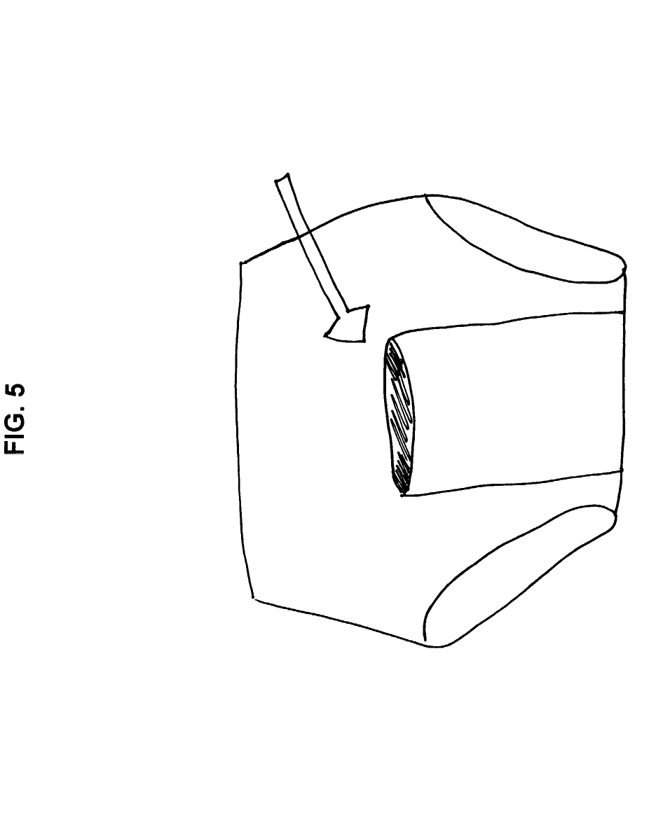

The present invention can be employed with any type of diaper or plastic training pants. When a person is using a cloth diaper with plastic training pants, the litmus paper is placed between the diaper and plastic training pants in a place where it is likely to get wet from the urine. FIG. 5 There are several methods to accomplish this. One method is to use a clear plastic training pant so that the litmus paper can be clearly observed for a color change when the cloth diaper gets wet with urine. FIG. 3 Another method is to design a clear plastic pouch that has perforated holes on one side of the pouch. FIG. 4 The other side of the pouch is made of a clear or transparent plastic without perforated holes. FIG. 3 A hole is cut into the portion of the plastic training pants that is most likely to get wet about the size of the pouch. The pouch is then adhered to the plastic training pants covering the hole with the perforated holes of the pouch on the inside and the clear plastic without the perforated holes on the outside. When used, a piece of litmus paper is placed in the pouch and then put on the person wearing the cloth diapers. FIG. 5 When the person wearing the diaper urinates, the urine will go through the perforated holes in the pouch and change the color of the litmus paper. FIGS. 3 & 4 When the litmus paper changes colors, the person responsible for changing the diaper will be able to observe the change of color through the clear plastic without perforated holes from the outside of the diaper. FIG. 3

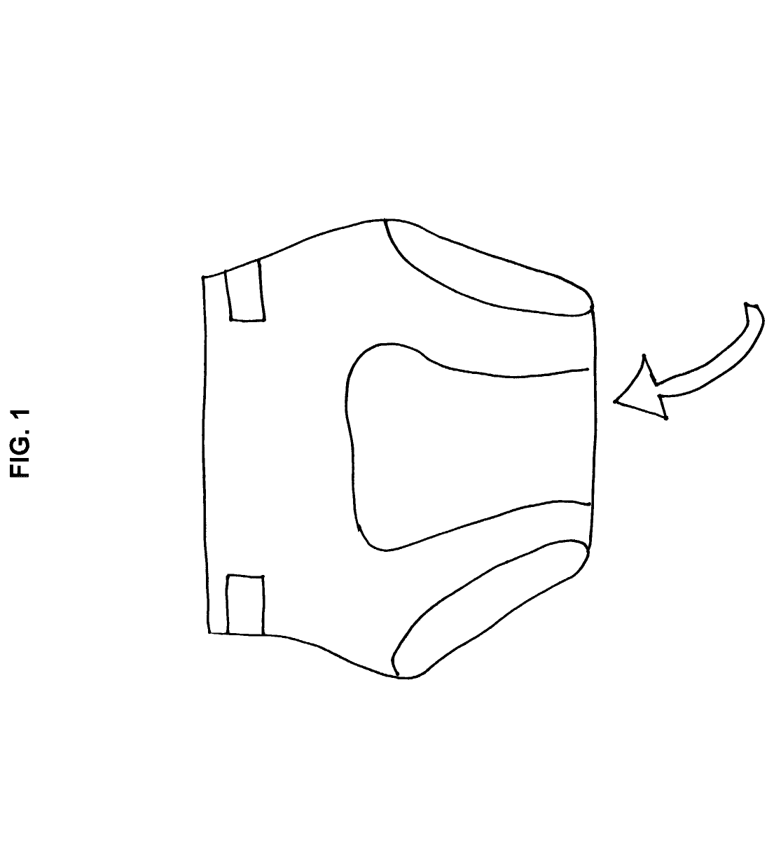

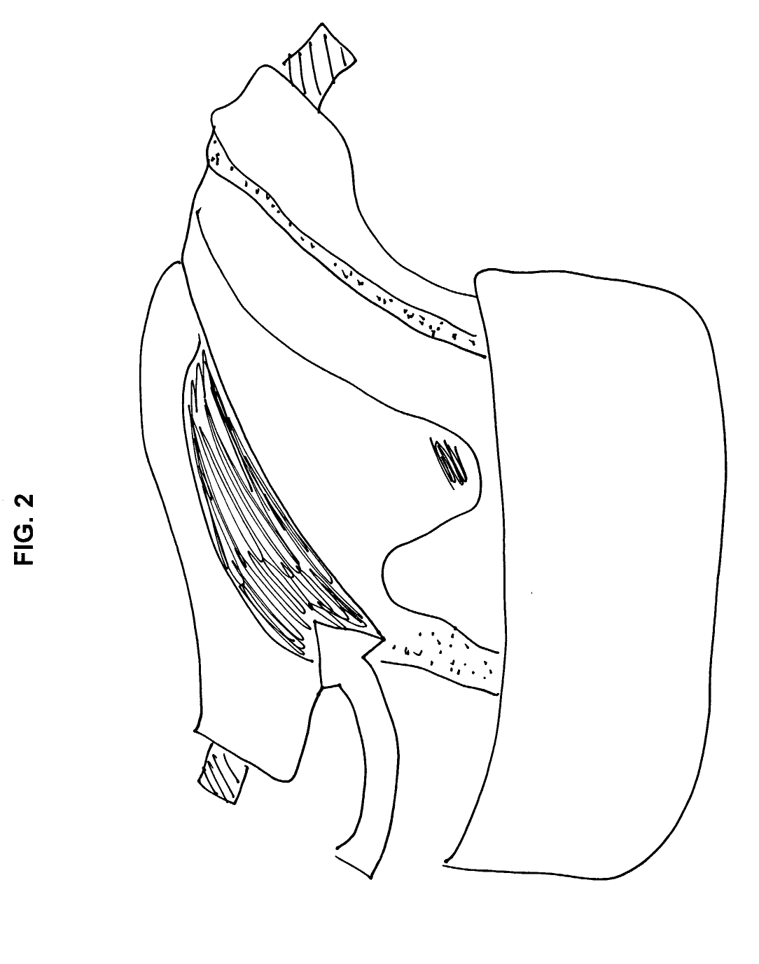

Most disposable diapers are made from wood cellulose fiber and polyacrylate material on the inside to absorb the urine and synthetic materials on the outside such as; polypropylene, polyester, and polyethylene to enhance fit and to prevent the diaper from leaking. The inventor recognizes that other materials are also used in the manufacturing of disposable diapers. For the purpose of the present invention, the inventor will refer to materials used by disposable diaper company in the manufacturing of diapers as either the absorbent inside material or the plastic outside. In this instance, the litmus paper will be placed between the absorbent inside and the plastic outside of the disposable diaper at a place where the diaper is likely to get wet with urine. FIG. 2. The plastic outside covering the litmus paper would ideally be made of clear or transparent plastic so that when the urine changes the color of the litmus paper, it can be noticed by the diaper changer. FIG. 1. Additionally, a portion of the plastic outside can be made with litmus paper without a clear or transparent plastic covering at a place on the disposable diaper where it will likely get wet with urine. FIG. 2.

Additionally, a maker of the early indicator color changing diapers and plastic training pants can substitute litmus paper with color changing or thermo chromic inks. The color changing ink can be place; on the absorbent inside, on material like paper or plastic between the absorbent plastic training pants and plastic outside or on the inside or outside of the plastic outside. In this instance, of the present invention, the color changing or thermo chromic inks will change color when a change in temperature occurs in the inks. The inks will change temperature when the urine comes in contact with it. There are primarily two types of color changing or thermo chromic inks liquid crystals and leucodye.

I claim:

1. A method of making an early indicator color changing diaper comprising of the steps of placing litmus paper between a cloth diaper and a plastic training pants at a place where it is likely to get wet with urine.

2. The method of claim 1 where the litmus paper is place in a pouch.

3. The method of claim 1 where the litmus paper is substituted with color changing ink.

4. A method of making an early indicator color changing diaper comprising of the steps of placing litmus paper placed between the absorbent inside and the plastic outside of a disposable diaper.

5. A method of claim 4 where the litmus paper is substituted with color changing ink.

6. A method of making an early indicator color changing diaper comprising of the steps of using litmus paper as the plastic outside of a disposable diaper.

7. A method of making an early indicator color changing diaper comprising of the steps of placing color changing ink on the plastic outside of a disposable diaper.

8. A method of claim 7 where the color changing ink is placed on the inside of the plastic outside of a disposable diaper.

9. A method of claim 7 where the color changing ink is positioned on a separate piece of plastic or paper placed between the absorbent inside and the plastic outside of a disposable diaper.

10. A method of claim 7 where the color changing ink is placed on the absorbent inside of a disposable diaper.

11. A method of making an early indicator color changing diaper whereby a chemical and/or thermal reaction caused by the heat or moisture of urine and/or other property creates some change to the color of some portion of a diaper providing a visual indicator that the diaper has urine.
